(19) United States
(12) Patent Application Publication
(10) Pub. No.: US 2007/0161906 A1
(43) Pub. Date: Jul. 12, 2007

(54) Method To Facilitate A Dermatological Procedure

(75) Inventors: Herbert D. Zeman, Memphis, TN (US); Gunnar Lovhoiden, Bartlett, TN (US); Roberto Kasuo Miyake, Sao Paulo (BR)

Correspondence Address: BUTLER, SNOW, O'MARA, STEVENS & CANNADA PLLC, 6075 POPLAR AVENUE, SUITE 500, MEMPHIS, TN, 38119, US

(73) Assignee: Luminetx Technologies Corporation, Memphis, TN (US)

(21) Appl. No.: 11/610,140

(22) Filed: Dec. 13, 2006

Related U.S. Application Data
(63) Continuation-in-part of application No. 10/386,249, filed on Mar. 11, 2003.
(63) Continuation-in-part of application No. 09/487,007, filed on Jan. 19, 2000, now Pat. No. 6,556,858.

(51) Int. Cl. A61B 6/00

(52) U.S. Cl. 600/473; 606/9

(57) ABSTRACT

The present invention relates to a process to facilitate dermatological procedures by illuminating a body part including at least one vein with a wavelength of light that is adsorbed by blood; producing a visible light image of said vein; coaxially projecting a computer-enhanced visible light image of the at least one vein onto the patients; and conducting a dermatological procedure on the patient. In one embodiment, the vein is a feeder vein. This method can be used to facilitate phlebectomy, sclerotherapy and botulinium toxin injection.

### CROSS REFERENCE RELATED APPLICATIONS TO

This application is a continuation-in-part of U.S. Pat. No. 6,556,858 filed Jan. 19, 2000, entitled Imaging System Using Diffuse Infrared Light and U.S. Ser. No. 10/386,249 filed Mar. 11, 2003 entitled Imaging System Using Diffuse Infrared Light (hereby specifically incorporated by reference in their entirety).

### BACKGROUND OF THE INVENTION

The present invention is generally directed to a system for illuminating an object with infrared light and producing a video image of the object based on reflected infrared light. The previously described imaging apparatus and system can be used to facilitate a dermatological procedure.

Telangiectasias can be subdivided into four classifications based on their macroscopic aspect, namely, simple or linear, arborizing, spider, and popular. In addition, they can also be classified according to the presence or absence of a feeder vein ( a vein with damaged valves that allows blood reflux into a smaller vein causing dilatation). For combined telangiectasias (CT) one or more feeder veins are present. For simple telangiectasias (ST), no feeder vein is present (Miyake H. et al. Tratamento das microvarizes e telangiectasias. In: Maffei F H, Lastria S, Yoshida W B, Rollo H A, editors. Doenças vasculares perifèricas. Rio de Janeiro: Medsi, 1563-80 2002:p.). The CT are located on the dermis and have feeder veins with damaged valves with reflux. These veins can be connected to the superficial and/or the deep venous system. Lack of such a connection characterizes the lesion as an ST, no matter what its appearance may be (Miyake H. et al. Surgical Treatment of Telangiectasias. Rev Hosp Clin Fac Dlin Med S Paulo 48:209-13 1993 and Miyake K. Tratamento A Laser de microvarizes. In: Maio M, editor. Tratado de medicina estética. S a̅o Paulo: Roca, 1055-72 2004:p.). Although both varicose veins and telangiectasias requiring treatment are clearly visible to the naked eye, feeder veins are often not apparent. The use of a device that enhances the ability to find feeder veins not visible to the naked eye may improve the treatment of telangiectasias.

### BRIEF SUMMARY OF THE INVENTION

The present invention relates to a process to facilitate dermatological procedures by illuminating a body part including at least one vein with a wavelength of light that is adsorbed by blood; producing a visible light image of said vein; coaxially projecting a computer-enhanced visible light image of the at least one vein onto the patients; and conducting a dermatological procedure on the patient. In one embodiment, the vein is a feeder vein. This method can be used to facilitate phlebectomy, sclerotherapy and botulinium toxin injection.

### DETAILED DESCRIPTION OF THE INVENTION

The invention provides a method to enhance the visibility of veins in dermatological procedures. Locating veins is critical for the treatment of varicose veins and telangiectasias. A subcutaneous vein that is invisible to the naked eye can be made easily discernible by the infrared imaging technology by projecting an enhanced image of subcutaneous veins onto the subject's skin. This can be accomplished by illuminating a body part, such as, for example, a leg with a wavelength of light that is adsorbed by blood. The vein in one embodiment is a feeder vein. A visible light image of the vein is produced and enhanced by processing and then coaxially projected onto the surface of the patient's skin undergoing the dermatological procedure.

The present imaging system in the preferred embodiment operates by illuminating the subject's skin with near infrared (NIR) or infrared light. This light penetrates skin and subcutaneous fat effectively because of the low absorption of these tissues in the NIR- or infrared wavelength range. The illuminated light is absorbed or scattered in the forward direction by blood, whereas it is scattered in all directions in skin and subcutaneous fat. Hence, blood reproduces as dark, whereas skin and fat appear lighter. The image reflected back from the subject is detected with a video camera. An infrared filter prevents any visible light from reaching the video camera. The resulting image is enhanced by a computer and then projected back onto the subject's skin with a projector using green light.

The present imaging system can be used in dermatological procedures to mark feeder veins and supplement the existing methods to treat the veins. Additionally, the present method can be used to access a vein in laser/sclerotherapy by increasing the likelihood feeder veins are treated. The terms the access means that the vein is physically contacted and surgically altered, removed or treated, or the like. Additionally, the present method can be used for vascular avoidance in certain dermatological procedures, such as botulinium toxin injection where it is undesirable to perforate a vein.

### EXAMPLE 1

Twenty-three consecutive subjects with telangiectasias that did not respond to laser and or sclerotherapy treatment were selected. Subjects with saphenous vein insufficiency and symptoms such as pain and/or edema were excluded from this study. Standard informed consent procedures were followed at all times. The study protocol conformed to the guidelines of the 12975 Declaration of Helsinki and was approved by our institutional review board.

The study was divided into five analyses: (1) diagnosing CT with the present imaging system (23 subjects); (2) comparison between the present imaging system and ultrasound (Ultrasound; two subjects); (3) marking feeder veins with or without the present imaging system (seven subjects); (4) phlebectomy of feeder veins using the present imaging system (seven subjects); and (5) laser and sclerotherapy with skin cooling guided by the present imaging system (Quantum D L (Lumenis, Inc., New York, N.Y.) 1,064-nm long-pulse laser treatments immediately followed by sclerotherapy, both techniques used with a cooler (Cryo5, Zimmer Elektromedizin, Neu-Ulm, Germany) (Miyake R K, et al. New Leg Veins Air Cooled Treatment Using 1064 nm Laser Combined with Sclerotherapy: Technique Description and One Year Follow-Up, Lasers Med Sci 18:522, 2003) that uses a high-velocity stream of cold air to numb the skin; 15 subjects).

Subjects were initially placed in dorsal decubitus and moved if necessary. The present imaging system was placed at the appropriate focal distance from the projector lens to the skin. The equipment head was placed perpendicular to the skin surface to maximize

A comparison was performed between the present imaging system and two types of Ultrasound machines, one portable (Pico, Medison, Sao Paulo, Brazil) and the other high resolution (Accuvix, Medison). First, subjects had their veins marked with ink dots using the present imaging system, and over each dot, ultrasound images were acquired. If the ultrasound was capable of detecting a vein, this mark was considered positive for ultrasound scanning. Positive and negative marks (where ultrasound detected no vein) were compared. The depth and diameter of feeder veins were measured with the ultrasound. Veins were also measured after removal.

Usually, feeder veins are marked before surgery with dots or dashes along their visible course. The ink usually used to indicate veins (“marks anything” style that is resistant to antiseptic) was found to alter the present imaging system images in preliminary tests before the beginning of the pilot study. Other markers were tested, and a thin-point black one that was soluble in alcohol was used. After veins were marked, the present imaging system was turned off, and other marks resistant to the antiseptic were put over the previous ones.

To improve the naked-eye view, many recommend using a combination of incandescent light, fluorescent light, and light from the sun (it is preferable to have large windows and schedule the procedure near noon). In contrast, use of the present imaging system requires less light to enhance the green image projected onto the skin. The number of marks without the present imaging system was counted. The machine was then turned on and veins were remarked. The number of marks before and after present imaging system use were compared.

Procedures were performed in the standard manner with the subject in a decubitus position and with antisepsis, placement of sterile surgical drapes, and anesthetic infiltration (2% lidocaine) performed. A local anesthesia technique device (The Wand Milstrone Scientific, Livingston, N.J.) was used, the same that we have been using the past three years. A future study testing the tumescent anesthesia is advisable. No IV sedation was used. After anesthesia; removal of feeder veins was initiated through successive mini-incisions employing a 40/12 needle, a No. 12 crochet hook for searching and catching, and delicate nippers. All the marked veins were laid on a table, an assistant measured the approximate vein size using a pachymeter, and the number of marks where the surgeon could not find a vein were counted. After that, sclerotherapy was performed to treat the telangiectasias and to test whether the veins were disconnected. The sclerosant solution used for all cases was 75% hypertonic dextrose. During sclerotherapy, lack of profuse leakage indicated a negative disconnection test. In this situation, the present imaging system was again employed in an attempt to find the remaining veins. Use of the present imaging system as a guide to finding veins was also analyzed.

Results were analyzed by comparing before and after photos, as well as by soliciting subject opinions. The sclerosant used was one of the most used in Brazil: 75% hypertonic dextrose. It is similar to the IV solutions commonly used at hospitals (5% and 25% but in a higher concentration). None of the subjects incurred an infection, and none showed any signs of being affected by the present imaging system. Physicians were able to identify feeder and varicose veins easily, with normal, dimmed, or no illumination in the room.

All 23 subjects were submitted to diagnosing CT with present imaging system. No subjects were excluded from the study because of failure to find feeder veins with the present imaging system. Photos taken with and without the present imaging system documented the presence and location of these veins. It was found that the present imaging system could also show the refilling process after decompression of CT in all of them. Physicians and subjects were able to see and discuss the treatment.

### EXAMPLE 2

A total of 75 marks were made by the present imaging system in two subjects. Of these 75 marks, 13 (17%) were also visualized by high-resolution ultrasound, and nine (13%) by portable Ultrasound, when placed over the marks. The deepest vein identified by the present imaging system was 0.8 mm in diameter and 7.8 mm deep. The present imaging system continued to visualize increasingly tiny veins until one 0.2 mm in diameter and 8.2 mm deep could not be identified. The smallest vein detected by the ultrasound measured 0.4 mm, and it could not find those shallower than 2.7 mm. These were of course easily visualized by the present imaging system.

Seven subjects who were scheduled for phlebectomy were marked with the naked eye. Adding them all, 103 marks were made. Employing the present imaging system, an additional 211 marks were added. Of this total of 314 marks, 67% were done only through the use of the present imaging system. In these seven subjects, the present imaging system indentified three times as many locations for marks as the naked eye. Because these subjects were CT, naked-eye visualization of feeder veins was either difficult or impossible. (Table 1).

TABLE 1

| | | | | | Percentage of |
|---|---|---|---|---|---|
| Subject | Naked- | | Total | Improve- | Marks Done |
| File | Eye | V-V-P | Number | ment | with the |
| Number | Marks | Marks | of Marks | Factor | V-V-P Only |
| 19655 | 15 | 15 | 30 | 2 | 50 |
| 25245 | 18 | 78 | 96 | 5.3 | 81 |
| 25849 | 7 | 16 | 23 | 3.3 | 70 |
| 26186 | 10 | 42 | 52 | 5.2 | 81 |
| 26187 | 9 | 26 | 35 | 3.9 | 74 |
| 26453 | 4 | 10 | 14 | 3.5 | 71 |
| 26488 | 40 | 24 | 64 | 1.6 | 38 |
| Total | 103 | 211 | 314 | 3.0 | 67 |

Seven subjects were submitted to phlebectomy, some of them in more than one area (a total of 16 areas). The marked feeder veins measured after removal averaged 0.96 mm. The smallest and biggest ones were 0.15 and 3 mm, respectively. After feeder vein phlebectomy, 13 areas tested positive for the disconnection test. Three of the 16 had a negative test. The present imaging system was then activated, and the remaining feeder veins identified by it were removed. The disconnection test immediately became positive in all three areas. On postoperative evaluation, the results for operated areas were considered good or excellent in all cases

### EXAMPLE 4

A total of 15 subjects with CT lesions were treated with laser and sclerotherapy guided by the present imaging system. Of these, nine reported a total or partial improvement of the lesion, four had no improvement, and two, so far, reported that the problem became worse. One was later treated by phlebectomy, with good results. The present imaging system was capable of guiding the laser treatments and also showing the effect of the laser (e.g., the vein collapsed partially and the present imaging system vein image became shorter and thinner) minutes after the laser shots.

While the foregoing description has set forth the various embodiments of the present invention in particular detail, it must be understood that numerous modifications, substitutions and changes can be undertaken without departing from the true spirit and scope of the present invention as defined by the ensuing claims. The invention is therefore not limited to specific preferred embodiments as described, but is only limited as defined by the following claims.

We claim:

1. A method to conduct a dermatological procedure on a patient, wherein the location of at least one vein facilitates said procedure comprising:
a) illuminating a body part including at least one vein with a wavelength of light that is adsorbed by blood;
b) producing a visible light image of said at least one vein;
c) coaxially projecting a computer-enhanced visible light image of said at least one vein on to said patient; and
d) conducting a dermatological procedure on said patient with knowledge of the location of the at least one vein.

2. The method of claim 1 wherein said dermatological procedure is phlebectomy.

3. The method of claim 1 wherein said dermatological procedure is sclerotherapy.

4. The method of claim 1 wherein said dermatological procedure is a botulinium toxin injection.

5. The method of claim 1 wherein said vein is a feeder vein not visible to the eye or detectable by ultrasound.

6. The method of claim 1 wherein said at least one vein is avoided during said dermatological procedure.

7. The method of claim 1 wherein said at least one vein is accessed during said dermatological procedure.
